(19) United States
(12) Patent Application Publication
(10) Pub. No.: US 2018/0234059 A1
(43) Pub. Date: Aug. 16, 2018

(54) LOW NOISE AMPLIFIER WITH HYBRID TERMINATION AND VARIABLE GAIN

(71) Applicant: General Electric Company, Schenectady, NY (US)

(72) Inventors: Naresh Kesavan Rao, Niskayuna, NY (US); Krishnakumar Sundaresan, Niskayuna, NY (US); Nanette Judith Gruber, Niskayuna, NY (US); Bruno Hans Haider, Niskayuna, NY (US)

(21) Appl. No.: 15/432,767

(22) Filed: Feb. 14, 2017

(51) Int. Cl. H03F 3/183; H03G 1/00; H03F 1/56; A61B 8/00; A61B 8/08

(52) U.S. Cl. CPC H03F 3/183; H03G 1/0088; H03F 1/56; A61B 8/4444; H03G 2201/106; A61B 8/461; H03F 2200/294; H03F 2200/222; A61B 8/52

(57) ABSTRACT

A system including a low noise amplifier is provided. The system further includes a coarse attenuation circuit coupled to an input of the low noise amplifier and configurable to attenuate an input signal by a coarse attenuation interval. The system also includes a fine attenuation circuit coupled in feedback with the low noise amplifier and configurable to attenuate the input signal by a fine attenuation interval, wherein the fine attenuation interval is less than the coarse attenuation interval.

### BACKGROUND

The subject matter disclosed herein relates to electronic devices and, more specifically, to electronic devices that employ a low noise amplifier with hybrid termination and variable gain.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Medical diagnostic ultrasound is an imaging modality that employs ultrasound waves to probe the acoustic properties of the body of a patient and produce a corresponding image. Generation of the ultrasound wave pulses and detection of the returning echoes is typically accomplished via a plurality of transducers located in an ultrasound probe. Such transducers typically include electromechanical elements capable of converting electrical energy into mechanical energy for transmission and capable of converting mechanical energy into electrical energy for receiving purposes. As will be appreciated, the ultrasound wave signals may exponentially decay as the signal propagates through the tissue of the patient. The amplitude of the signal is at its highest near the surface of the skin where little amplification is used, but as the signal travels deeper, the signal's amplitude decreases. Therefore, further amplification may be used to increase the amplitude of the signal as it travels further into the body. The goal of a time dependent amplifier is to try to maintain a uniform envelope at the output even though the input signal envelope is exponentially decaying. It is now generally recognized that enhanced electronics are desired that achieve the characteristic signal envelope commonly used in ultrasound electronics and that closely match the impedance of the probe to reduce reflections which may cause image artifacts to suit a variety of health modality applications.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the present disclosure. Indeed, the disclosed techniques may encompass a variety of forms that may be similar to or different from the embodiments set forth below.

In accordance with one embodiment, there is provided a system comprising a low noise amplifier. The system further comprises a coarse attenuation circuit coupled to an input of the low noise amplifier and configurable to attenuate an input signal by a coarse attenuation interval. The system also comprises a fine attenuation circuit coupled in feedback with the low noise amplifier and configurable to attenuate the input signal by a fine attenuation interval, wherein the fine attenuation interval is less than the coarse attenuation interval.

In accordance with another embodiment, there is provided a system comprising an ultrasound probe. The ultrasound probe comprises a low noise amplifier. The ultrasound probe further comprises an R2R ladder coupled to an input of the low noise amplifier and configured to provide coarse gain control through the low noise amplifier. The ultrasound probe also comprises a resistive feedback network coupled in feedback with the low noise amplifier and configurable to provide fine gain control through the low noise amplifier. The system further comprises a console communicatively coupled to the ultrasound probe and configured to facilitate ultrasound image collection and processing in conjunction with the ultrasound probe.

In accordance with another embodiment, a method for performing an ultrasound scan is provided. The method comprises initializing a fine gain correction circuit and a coarse gain correction circuit to provide maximum attenuation of a scan signal. The method further comprises adjusting, using the fine gain correction circuit, an attenuation of the scan signal by a fine attenuation increment until a total attenuation of the scan signal by fine attenuation increments is approximately equal to a coarse attenuation increment. The method also comprises adjusting, using the coarse gain correction circuit, the attenuation of the scan signal by the coarse attenuation increment.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a block diagram illustrating an ultrasound system, in accordance with an embodiment of the present disclosure;

FIG. 2 is a more detailed block diagram illustrating additional features of the probe of FIG. 1, in accordance with an embodiment of the present disclosure;

FIG. 3 is a block diagram of a portion of an analog front-end of the digital probe of FIG. 2, in accordance with an embodiment of the present disclosure;

FIG. 4 is a schematic diagram of the architecture of the R2R ladder and resistor feedback network of FIG. 3, in accordance with an embodiment of the present disclosure;

FIGS. 5-7 illustrate an iterative process of coarse and fine gain adjustments through the system, during a scan sequence, in accordance with an embodiment of the present disclosure; and

FIG. 8 is a flow chart summarizing the scan sequence described with regard to FIGS. 5-7, in accordance with an embodiment of the present disclosure.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

Some embodiments of the present disclosure generally relate to a low noise amplifier (LNA) with hybrid termination and variable gain implemented in a probe for use in ultrasound imaging. The LNA may enable a large dynamic range, low noise, and low power with termination that matches the probe impedance to reduce reflections which may cause image artifacts. The LNA and related components described herein may be included on an application specific integrated circuit (ASIC) that also employs a hybrid implementation using digital assisted analog and close integration with system software.

Generally, implementing termination to avoid reflections involves matching input impedance to probe impedance. This is generally achieved by using either a passive termination by employing a shunt resistor, or by using an active device, such as an input impedance of the LNA, also referred to as active termination. It should be appreciated that passive termination using a resistor, though simple and having a very large dynamic range, is inherently noisy. In contrast, active termination generally exhibits low noise but also has tradeoffs between desired dynamic range and power. Some embodiments of the present disclosure may include a hybrid approach utilizing both active and passive techniques to achieve termination based on the magnitude of the signal from the probe. When the signal is large, passive termination in the form of a resistor structure ladder (e.g., R2R ladder) may be used, as described in detail below. When the signal is small, the signal may bypass the R2R ladder entirely and is processed by the LNA directly, ensuring low noise with active termination. The ASIC provides a large dynamic range by attenuating signals coarsely using the R2R ladder. Fine gain control may be enabled by manipulating the feedback gain of the LNA such that the gain varying as a function of the amplitude is smooth, as described in detail below. By employing a hybrid scheme utilizing both active and passive elements to match input impedance to probe impedance to provide low noise measurements over a large dynamic range, an improved system may be achieved. In accordance with embodiments of the disclosure, the disclosed hybrid scheme leverages the advantages of both passive and active termination and provides variable gain to achieve a low power, low noise system with a large dynamic range with proper termination and low second harmonic distortion.

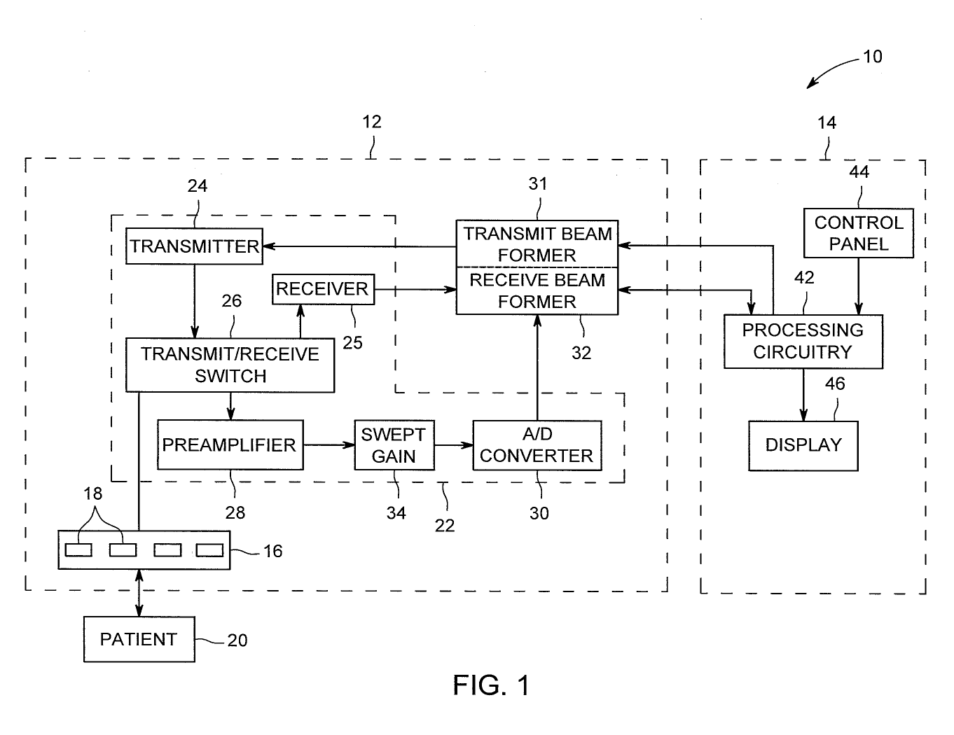

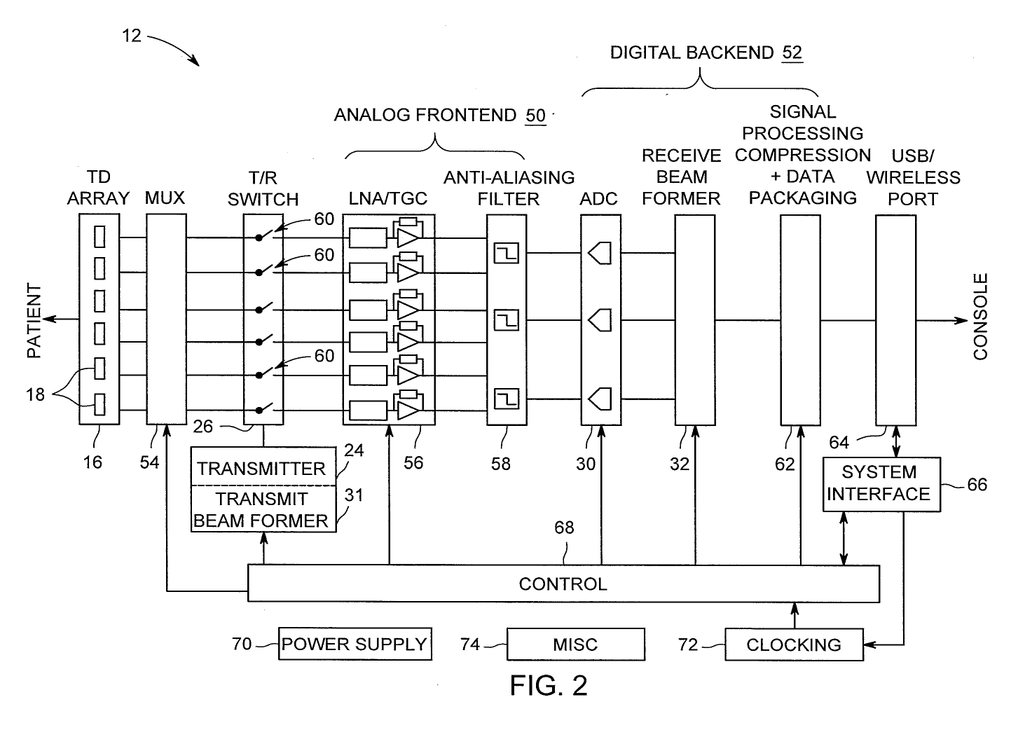

Turning now to the drawings, FIG. 1 is a block diagram illustrating an embodiment of an ultrasound system 10 capable of implementing the presently disclosed low noise amplifier with hybrid termination and variable gain. In the depicted embodiment, the ultrasound system 10 is a digital acquisition and beam former system, but in other embodiments, the ultrasound system 10 may be any suitable type of ultrasound system, not limited to the depicted type. The illustrated ultrasound system 10 includes an ultrasound probe 12 and a console 14. The probe 12 may be coupled to the console 14 by any suitable technique for communicating image data and control signals between the probe 12 and the console 14 such as wireless, optical, coaxial, etc. A more detailed block diagram of the probe 12 will be described with regard to FIG. 2.

The probe 12 of FIG. 1 includes a transducer array 16 having transducer elements 18. Each individual transducer element 18 is generally capable of converting electrical energy into mechanical energy for transmission and mechanical energy into electrical energy for receiving. As will be appreciated by those skilled in the art, the transducer elements 18 may be fabricated from materials, such as, but not limited to lead zirconate titanate (PZT), polyvinylidene difluoride (PVDF) and composite PZT. It should be noted that the transducer array 16 is configured as a two-way transducer capable of transmitting ultrasound waves into and receiving such energy from a subject or patient 20 during operation when the probe 12 is placed in contact with the patient 20. In one embodiment, during operation of the ultrasound system 10, an image is created using a pulse echo method of ultrasound production and detection. In this embodiment, a pulse is directionally transmitted into the patient 20 via the transducer array 16 and then is partially reflected from tissue interfaces that create echoes that are detected by the transducer elements 18.

More specifically, in transmit mode, the transducer array elements 18 convert the electrical energy from the probe 12 into ultrasound waves and transmit the signals into the patient 20. In receive mode, the transducer array elements 18 convert the ultrasound energy received from the patient 20 (reflected signals or echoes) into electrical signals for transmission and processing by the probe 12 and console 14 to provide an ultrasound image that may be analyzed. The number of transducer elements 18 in the array 16 and the frequencies at which the transducer elements 18 operate may vary. For instance, the transducer array 16 may include a number of elements in the range of 32-512 transducer elements 18. Further, the transducer array 16 may operate at frequencies in the range of 1 MHz-15 MHz, for example. As will be appreciated, other array sizes and operational frequencies may be utilized, depending on the application.

Each transducer element 18 is associated with its respective transducer circuitry 22. That is, in the illustrated embodiment, each transducer element 18 in the array 16 may have corresponding transducer circuitry 22 each including a corresponding transmitter 24, a receiver 25, a transmit/receive switch 26, a preamplifier 28, and an analog to digital converter (ADC) 30. For example, in an embodiment in which the transducer array 16 includes 128 transducer elements 18, there may be 128 sets of transducer circuitry 22, one for each transducer element 18. In alternative embodiments, certain or all blocks of the transducer circuitry 22 may be shared by more than one transducer element 18, such that a 1-to-1 correspondence of blocks to transducer elements 18 is not employed.

The probe 12 may include a variety of other imaging components that when used in conjunction with the components of the console 14, enable image formation with the ultrasound system 10. For instance, the probe 12 may include a transmit beam former 31 and a receive beam former 32, represented collectively by block 31/32 (“beam former 31/32”) in FIG. 1. The beam former 31/32 is capable of providing digital focusing, steering, and summation of the beam. In this way, the beam former 31/32 may control and generate electronic delays in the transducer array 16 to achieve the desired transmit and receive focusing, as will be described in greater detail below with regard to FIG. 2. In addition, the transducer circuitry 22 of the probe 12 may include a swept gain 34 to reduce the dynamic range of the received signals prior to digitization.

During operation, the transmitter 24 provides a pulsed electrical voltage suitable for excitation of the transducer elements 18 and may adjust the applied voltage to control the output transmit power. The transmit/receive switch 26 is synchronized with the transmitter 24 and is capable of isolating the high voltage (e.g., approximately 150 V) used for pulsing from the amplification stages during receiving cycles. In some embodiments, the probe 12 may include a handle portion (e.g., a grooved section designed for gripping) that facilitates use by an operator, such as a medical technician. Additionally, it should be noted that the probe 12 may be manufactured to take on any of a number of geometries, such as a t-shape, a rectangle, a cylinder, and so forth. As will be discussed in detail below with regard to FIG. 2, the probe 12 may include a number of additional elements to optimize the usage of the ultrasound system 10, in accordance with embodiments of the present disclosure.

As previously described, the probe 12 is coupled to the console 14 to facilitate image collection and processing. As will be appreciated, the console 14 may include a number of elements to control operation of the probe 12 and facilitate the production of images that may be viewed by clinicians and/or patients 20. For instance, the console 14 of FIG. 1 includes processing circuitry 42, a control panel 44 and a display 46. In certain embodiments, the console 14 may include additional elements not shown in FIG. 1, such as additional data acquisition and processing controls, additional image display panels, multiple user interfaces, and so forth. Further, the console 14 may be a mobile device, such a as smart phone.

During a detection or receive mode, the processing circuitry 42 in the console 14 receives matrices of digital data representing reflection signals returned from tissue interfaces within the patient 20 during a pulse-echo data acquisition method. These matrices of digital data, or processed versions of these matrices, are transmitted to the processing circuitry 42 from the probe 12. During an acquisition process, the transducer array 16 of the probe 12 is positioned on the patient 20. The transmitter 24 transmits ultrasound energy into the patient 20 via the transducer elements 18 of the transducer array 16, and the receiver 25 receives data from the array of transducers 26 corresponding to matrices of data representing reflection signals returned from tissue interfaces within the patient 20. Once the receiver 25 receives data from the transducer array 16 corresponding to matrices of data representing reflection signals returned from tissue interfaces within the patient 20 during data acquisition, these matrices of data may be processed and communicated to the console 14 via a processed electrical signal. In some embodiments, the processed electrical signal may correspond directly to the matrices of data. However, in other embodiments, the processed electrical signal may correspond to compiled data received from more than one transducer element 16 and/or data that has been processed, for example, to reduce or eliminate signal noise.

As will also be appreciated, in certain embodiments, the transducer elements 18 may be voltage biased when receiving echoes back from the patient 20. That is, the transducer elements 18 may be pre-charged to a certain voltage (e.g., 1 V, 2 V) prior to receiving signals back from the patient 20 such that all received signals take on a positive value. The foregoing feature may have the effect of simplifying electrical circuitry associated with the receiving cycle in certain embodiments, as will be appreciated.

Once received by the console 14, the electrical signal is transferred to the processing circuitry 42 for processing. Accordingly, the processing circuitry 42 may include memory, which may be volatile or non-volatile memory, such as read only memory (ROM), random access memory (RAM), magnetic storage memory, optical storage memory, and so forth, for storing and/or processing the signals. Once processed, the matrices of data may be utilized to produce an image of the patient's anatomy that is displayed on the display 46 in accordance with operator selections and parameters input via the control panel 44, which may include any user interface, such as a keyboard. As will be appreciated, any number of additional elements may be incorporated into the console 14 to aid in the control, processing, analysis and production of ultrasound images.

Turning now to FIG. 2, a more detailed embodiment of the digital probe 12 of FIG. 1 is illustrated. As will be described in greater detail below, the probe 12 includes features that provide a hybrid scheme utilizing both active and passive elements to match input impedance to probe impedance to provide low noise measurements over a large dynamic range, based on signal size. The probe 12 includes an analog front end (AFE) 50 and a digital back end (DBE) 52. As will be appreciated, the transmitter 24 imposes a high voltage (HV) pulse sequence on the transducer elements 18 to create an ultrasound wave in the patient 20 and the AFE 50 receives analog signals from the patient 20. The probe 12 may include additional elements for system interface, storage, clocking, power supply, control, etc. as will be described further below. It will be appreciated that the block diagram of FIG. 2 is a simplified representation of the probe 12 and that additional elements may also be included within the probe 12.

In the illustrated embodiment, the AFE 50 includes a low noise amplifier (LNA)/time gain control (TGC) block 56 and an anti-aliasing filter (AAF) 58. As previously described, the transducer array 16 includes a number of transducer elements 18 capable of receiving electrical signals from a console 14 and converting them into ultrasound waves to be directed into a patient 20. The transducer array 16 is further capable of receiving echo ultrasound signals reflecting back from various tissue structures in the patient 20 and converting the echo signals to electrical signals for image production. The transmit/receive switch 26 includes a number of individual switching elements 60, corresponding to the number of transducer elements 18. The switching elements 60 may comprise a diode bridge to block high voltage signals during the transmit mode, for example. As will be appreciated, the switching elements 60 are open while the probe 12 is in the transmit mode (i.e., transmitting ultrasound signals into the patient 20) and closed while the probe 12 is in the receive mode (i.e., receiving echo signals reflected from tissue structures in the patient 20). By opening the switching elements 60 during the transmit mode, the AFE 50 is isolated from the high voltage (e.g., 200 volts) that may be used to transmit the electrical signals into the patient 20.

Because the number of transducer elements 18 in the array 16 may be greater than the number of transmitters 24 and receivers 25 (FIG. 1) a plurality of multiplexors in a multiplexor block 54 may also be provided. Each multiplexor in the multiplexor block 54 may be a high voltage element capable of handling large voltage swings (e.g., 200 volts peak-to-peak) for a particular ultrasound application. The multiplexors may be used to connect a specific transducer element 18 to a specific transmitter/receiver pair, for instance, such that the system 10 can dynamically change the active transducer aperture over the available transducer element array 16. The multiplexor block 54 may be utilized to reduce the complexity of the transmit/receive hardware. As will be appreciated, the multiplexor block 54 may also be eliminated such that each transducer element 18 couples directly to a switching element 60.

During transmission, the transmit beam former 31 determines the delay pattern and pulse train that set the desired transmit focal point for the transmitters 24. As previously described, the outputs of the transmit beam former 31 are then amplified by high voltage transmit amplifiers that drive the transducer elements 18 of the array 16. The amplifiers may be controlled to shape the transmit pulses for better energy delivery to the transducer elements 18. Typically, multiple transmit focal regions (zones) are used during a scan. In other words, the field to be imaged is deepened by focusing the transmit energy at progressively deeper points in the body of the patient 20 such that the transmit energy can be amplified as the signal travels deeper into the body, to adjust for the signal's attenuation as it travels into the body and the echoes return to the transducer elements 18. For instance, the transmit signal may attenuate at about 1 dB/cm/MHz.

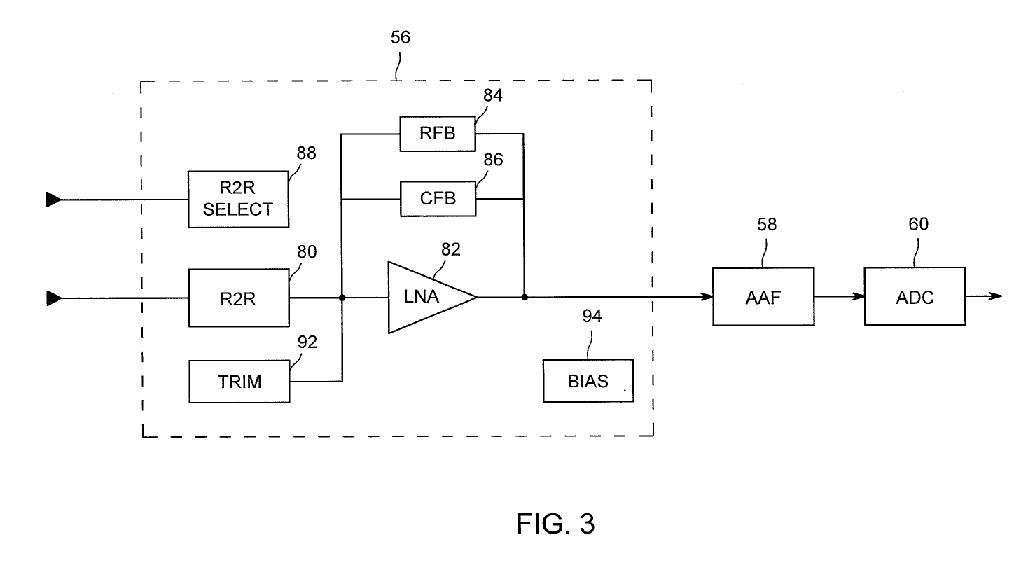

In accordance with the presently described techniques, the probe 12 also includes a low noise amplifier (LNA)/time gain control (TGC) block 56. The LNA/TGC block 56 includes a number of individual LNA/TGC elements to provide for a unique LNA configuration with elements for both coarse and fine gain control and to allow for the dynamic matching of input impedance to probe impedance over a large dynamic range. By incorporating both active and passive elements to match input impedance to probe impedance to provide low noise measurements over a large dynamic range, an improved system may be achieved. The elements of the unique LNA/TGC block 56 which may be implemented on an application specific integrated circuit (ASIC), for instance, will be described in detail below with regard to FIG. 3.

The AFE 50 also includes an anti-aliasing filter (AAF) 58. Generally, the AAF 58 functions as a low pass filter. Specifically, the AAF 58 prevents high-frequency noise and extraneous signals that are beyond the normal maximum imaging frequencies from being aliased back to baseband by the ADC 30. The AAF 58 may be adjustable, in certain systems. In certain embodiments, the AAF 58 may include one or more Butterworth filters or one or more higher-order Bessel filters, for instance.

As will be appreciated, the AFE 50 communicates with the DBE 52. The DBE 52 may include ADCs 30, a receive beam former 32 and a signal processing, compression and data packaging block 62. The ADC 30 may be a 12-bit device, for instance. The ADC 30 may include a number of individual analog-to-digital converters for converting the analog signals from the AFE 50 to digital signals for processing and image production. The ADC 30 is generally selected to provide rapid dynamic range at acceptable power levels. The ADC 30 also limits the signal-to-noise ratio of the receiver 25 (FIG. 1). The ADC 30 provides the ultrasound signal echoes from the patient to the receive beam former 32. As will be appreciated, the receive beam former 32 may be a digital beam former. In alternative embodiments, the receive beam former 32 may be an analog beam former that is arranged to receive signals directly from the AFE 50 and provide beam formed analog signals to the ADC 30. As will be appreciated, the DBE 52 may additionally include a number of other functional elements, such as components provided for digital signal processing, data compression, data packaging, filtering, storage, etc., as indicated collectively by block 62.

The probe 12 may include a number of other elements to provide for improved functionality and versatility. For instance, the probe 12 may include a wireless or wired port (e.g., a USB port 64) to provide digital communication from the probe 12 to the console 14. A system interface 66 may also be provided on the probe 12 to allow for more user-friendly usage of the probe 12 and the ultrasound system 10. Control of the probe 12 is generally represented by the control block 68 which may include hardware and/or software components to control the image acquisition process and the various components of the probe 12. The probe 12 may also include a power supply 70, such as a battery, for instance. The power supply block 70 may also provide voltage regulation and distribution for the probe 12. The probe 12 may also include clocking circuitry 72 for controlling the timing of transmit and receive signals, as well as scan sequencing. The probe 12 may also include other components designed to provide a versatile, mobile, sensitive and secure system, such as drop sensors, thermal sensors to protect a patient from exposure to overheated elements, authentication and security to provide patient confidentiality, etc., as represented collectively by the block 74.

Turning now to FIG. 3, a more detailed block diagram of certain components of the disclosed LNA/TGC block 56 of the AFE 50 is illustrated. As will be appreciated, only a single input path of the LNA/TGC block 56 is illustrated. That is, the elements of the LNA/TGC block 56 associated with each input path correspond to one of the transducer elements 18 of the transducer array 16. Accordingly, the illustrated components may be duplicated for each of the transducer elements 18 of the array, depending on the design of the probe 12. The elements of the LNA/TGC block 56 may be provided on an ASIC and controlled by software of the system 10. As previously described with regard to FIG. 2, the outputs of the LNA/TGC block 56 may be transmitted to the AAF 58 and ADC 60.

Each circuit of the LNA/TGC block 56 includes an R2R ladder 80 coupled to a low noise amplifier (LNA) 82 (e.g., class AB) with a resistive feedback (RFB) network (RFB) 84, capacitive feedback network (CFB) 86 and an R2R select register 88. The LNA/TGC block 56 circuit architecture incorporates dual-action attenuation and amplification functions to enable the desired characteristic signal envelope used in the system 10. As described below, the dual-action function may be enabled by the attenuating R2R ladder 80 and an amplifying switched resistor feedback structure implemented by the LNA 82 and the RFB network 84. This may result in an output signal envelope which is gradually increasing in amplitude for a constant input signal, and therefore mimics in reverse time order the near and far-field intensity of ultrasound signals as the signal travels through the body of the patient 20 (FIG. 1). As also described below, the capacitive feedback network 86 includes a number of switched capacitor feedback combinations which allow for adjustable bandwidths utilizing varying switch settings. Further, based on the design of the R2R ladder 80 and the inclusion of multiple parallel blocks in the R2R ladder 80, the effective resistance in the R2R ladder 80 is programmable, which allows for variable matching to the probe impedance. The R2R select register 88 may be used to select the proper impedance through the R2R ladder 80, depending on the application. By selecting, via software, the number of R2R blocks that are placed in parallel, the impedance of the probe may be closely matched to suit a variety of health modality applications such as cardiac, abdominal, women's health, vascular, and high frequency linear imaging.

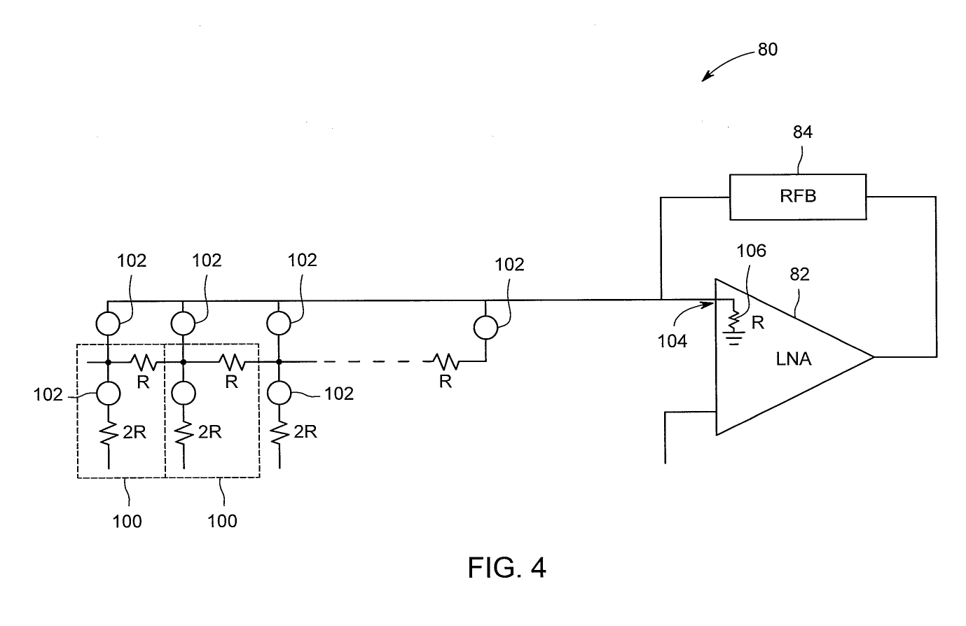

As will be described in greater detail with reference to FIGS. 4-8, during operation, the LNA/TGC block 56 enables amplification/attenuation control by utilizing the R2R ladder 80 in conjunction with the LNA 82. Coarse gain correction is performed by controlling the most significant bits (MSBs) feeding the R2R ladder 80 (i.e., switching R2R blocks in and out of the signal path into the LNA 82, using the R2R select register 88). This generally enables attenuation in large increments. Fine gain correction may be performed by digitally controlling the RFB network 84 to decrease the resistance by switching in additional small valued resistors, like a digital potentiometer. As will be appreciated, fine gain correction may be implemented using a shift register or direct control. The LNA/TGC block 56 may also include an LNA trim circuit 94. The trim circuit 92 may be provided to trim the offset voltage to a smaller resolution (e.g., 10 bits). When a 0 V DC input is applied to the LNA 82, the trim bits may be adjusted to adjust the output voltage to as close to 0V as possible to maximize dynamic range of the LNA 82 and ADC 30. The bias circuit 94 provides references and bias potentials for powering the LNA/TGC block 56 and might have further digital controls for further reducing noise if the application requires it.

The capacitive feedback network 86 is responsible for providing four switched capacitor feedback combinations which allow for adjustable bandwidths. The bandwidth of the LNA 82 is adjustable from 12 MHz, 15 MHz, 20 MHz, and 30 MHz, by varying the feedback capacitance through a series of switch settings for additional filtering. However, as will be appreciated, the primary filter for limiting the bandwidth of the LNA 82 is the AAF 58.

In general, the R2R ladder 80 includes multiple R2R blocks arranged in parallel. These blocks can be added or removed from the signal path through a control select signal from the R2R select register 88. By selecting the number of R2R blocks that are utilized at a particular time, the value of the input impedance may be altered to more closely match the characteristic impedance of the probe 12. For example, since the resistance in the R2R ladder 80 should match the probe impedance, if a probe impedance of 89 ohms is desired as in the case of cardiac applications, then a maximum number of resistors (R2R blocks) may be selected such that they are placed in parallel in the R2R ladder 80. Conversely, if a resistance of 134 ohms is desired, as in the case of a vascular application, then fewer R2R blocks may be selected such that they are placed in parallel. The function of an R2R select register 88 is to select the appropriate resistance which matches the impedance of the probe 12 for a variety of health modalities, such as cardiac, women's health, vascular, abdominal, and high frequency linear imaging.

The R2R select bus connects a specified number of R2R ladder blocks in parallel. In the presently described embodiment, the R2R ladder 80 may enable gain attenuation in 6 dB increments starting from −30 dB (or full attenuation) down to 0 dB or no attenuation. That is, each R2R block in the R2R ladder 80 increments the attenuation by 6 dB, which corresponds to a change in amplitude ratio by a factor of 2 (or equivalently, a change in the power by a factor of 4). However as will be appreciated, the R2R ladder 80, and the R2R blocks may be designed to provide for adjustment at other desired increments, as well, depending on the application.

Referring now to FIGS. 4-8, the operation of the LNA/TGC block 56 is described. Specifically, the operation of the R2R ladder 80 and the RFB network 84 of the LNA/TGC block 56 is described from the beginning of a scan sequence, wherein the signal is large and passive termination is employed through the R2R ladder 80 and eventually a small signal and active termination is employed using the input impedance of the LNA 82. The input impedance of the LNA 82 is represented by the resistor 106, having a value of R ohms. The LNA 82 and its internal elements and the feedback across the LNA 82, in conjunction, provide the input termination of the LNA 82. As will be appreciated, the R2R ladder 80 should also be properly terminated. This termination is provided by the input termination of the LNA 82, as well. Thus, the R2R ladder 80 attenuates the large signals and the R2R ladder 80 is terminated by the input impedance 106 of the LNA 82. For signals which are far field, the R2R ladder 80 is not utilized and the probe 12 is directly terminated by the input impedance 106 of the LNA 82. The coarse gain adjustments (using the R2R ladder 80) and the fine gain adjustments (using the RFB network 84) are also described.

As illustrated in FIG. 4, the R2R ladder 80 includes a number of R2R blocks 100. Each R2R block 100 includes a resistor R and a resistor 2R. The value of R will be chosen to match the impedance of each transducer element 18. For instance, if the impedance of the transducer elements 18 in array 16 is 100 ohms, the value of the resistor R will also be 100 ohms and the value of the resistor 2R will be 200 ohms. As described above, in the embodiment described herein, each R2R block 100 corresponds to a 6 dB step in attenuation when added to the input path. Each R2R block 100 can be added or removed from the input path by using the R2R select register 88 and the various combinations of the switches or taps 102. When the taps 102 are selected (i.e., opened), a signal path to the first input 104 of the LNA 82 is provided. By adding a R2R block 100 to the path, the attenuation is coarsely adjusted in the larger increments (here, 6 dB).

Between each of the coarse adjustments provided by the addition (or removal) of R2R blocks 100 from the signal path, fine adjustments are made through the RFB network 84. The RFB network 84 includes a number of feedback resistors which may be opened to slowly (fine adjustment) decrease the gain in smaller increments. For instance, in one embodiment, the RFB network 84 includes 64 resistors in feedback. In one embodiment, as each resistor in the RFB network 84 is opened, the gain through the feedback loop decreases by 0.3 dB. This allows for fine attenuation of the gain. The feedback resistor is chosen such that the output signal is approximately 1 Vpp. As previously discussed, different ultrasound probes are used typically used based on the type of scan required. For example, cardiac or women's health probes have different resistance matching requirements. They can be as low as 88 ohms or as high as 136 ohms. Depending on the probe, there is a minimum feedback resistor across the LNA 82. For example, if the probe is 100 ohms and the input signal is 1V, the voltage at the input of the LNA is −30 dB of 1 V or 31 mV. The resistance through the RFB network 84 is chosen such that the gain—the output of the LNA 82 (0.5 V) divided by the input of 31 mV—times the series resistance of 100 ohms is equal to the feedback resistor, or 1.58K ohms. As the signal decreases, the gain is increased by 0.3 dB until the RFB network 84 increases to the next 6 dB tap or approximately 20 steps. At this point, the appropriate taps 102 are set such that the R2R ladder 80 is lowered to −24 dB of attenuation, and the RFB network 84 is reset back to 1.58 Kohms, where it restarts the 20 step increment. This process is repeated until the R2R ladder 80 is set to 0 dB of attenuation (or complete signal passing) with the RFB network 84 reset to 1.58 Kohms and 20 step incrementing.

As will be appreciated, while fine attenuation adjustments have been described in the illustrated example as being approximately 0.3 dB increments, other fine increments may be employed. For instance, the fine attenuation adjustments may be anywhere in the range of approximately 0.1 dB to 1.0 dB (such as 0.3 dB). Also, while the present embodiment is described with fine attenuation adjustments made in discrete steps, a digital-to-analog converter could also be used to provide smooth fine attenuation adjustments, rather than discrete steps.

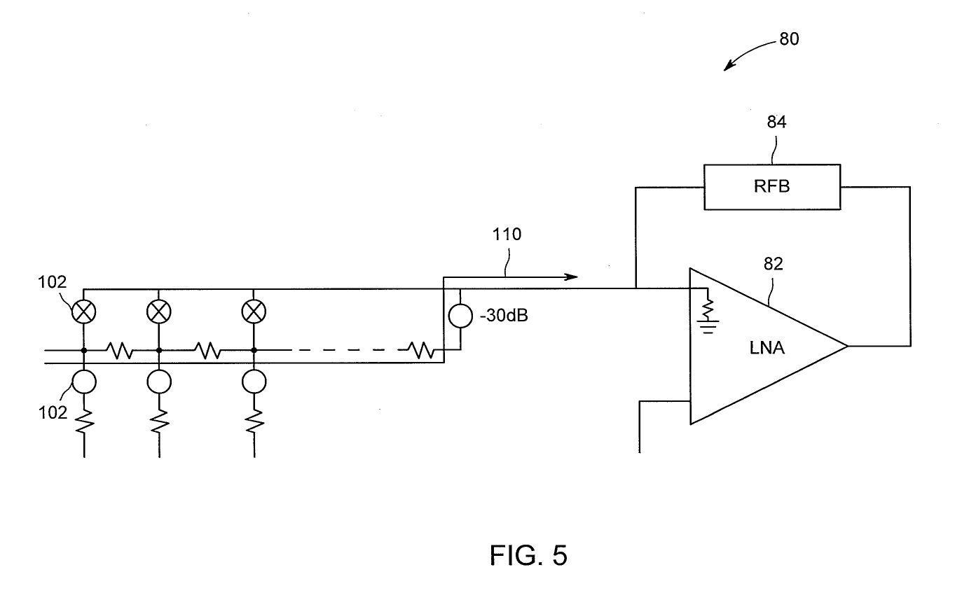

Thus, the LNA/TGC block 56 functions by coarsely attenuating the gain in increments of approximately 6 dB using an R2R ladder 80, and successively connecting switched resistors in feedback (within the RFB network 84) with the LNA 82 in order to increment the gain by approximately 0.3 dB. For example, FIG. 5 illustrates the beginning of a scan sequence. As the sequence begins, the taps 102 of the R2R ladder 80 are initially set at −30 dB since the amplitude of the signal is highest near field or at the skin surface. That is, certain taps 102 are opened (as represented by an “O” in FIG. 5. Other taps 102 are left closed (as represented by an “⊗”). By opening the taps 102, a signal path 110 is created. FIG. 5 illustrates the setting to allow a maximum resistance through the R2R ladder 80, such that the attenuation of −30 dB can be achieved. The fine gain adjustment of 0.3 dB steps in amplification begins until a count of 20 steps has been achieved or a total interval equal to the smallest interval of the coarse delay (here, 6 dB), as previously described. The next tap 102 of the R2R ladder 80 is then selected to remove a R2R block 100 from the signal path such that an attenuation of −24 dB is set, and the fine gain increment is again repeated for an interval of 6 dB through the RFB network 84. This process continues such that when the last tap 102 of the R2R ladder 80 is set to 0 dB or no attenuation, the signal amplitude is at its highest in the far field or when it has penetrated deep inside the body of the patient 20. The effective signal envelope is determined by the operation of the fine feedback resistor network gain increments of the RFB network 84 and coarse R2R ladder gain attenuation of the R2R ladder 80.

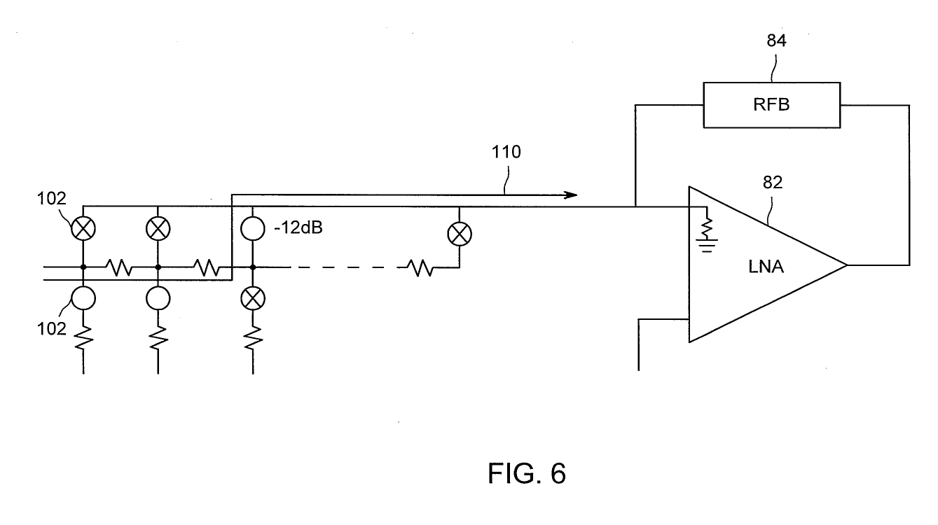

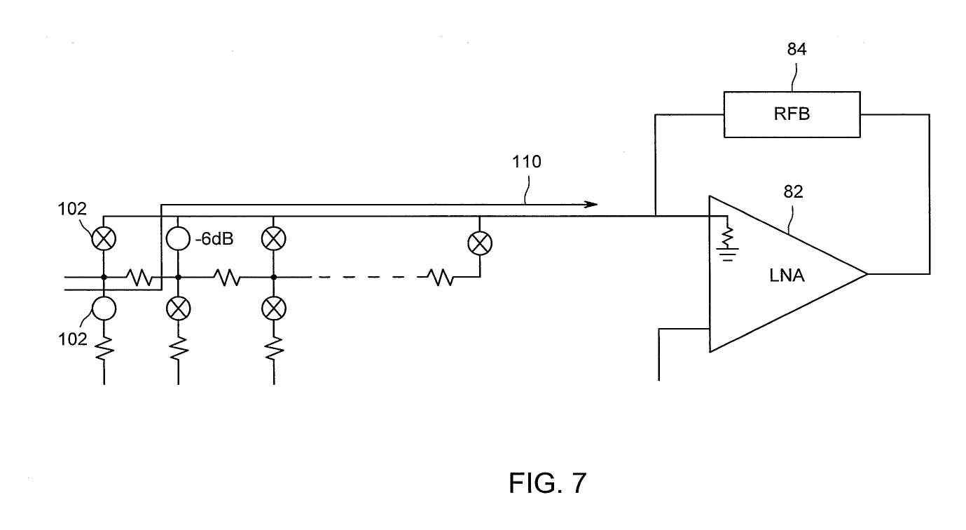

FIGS. 6 and 7 illustrate other configurations of the R2R ladder 80 at a later time in the scan sequence when the signal has traveled deeper into the body of the patient 20. Specifically, FIG. 6 illustrates the setting of the taps 102 to achieve an attenuation of −12 dB. FIG. 7 illustrates the setting of the taps 102 to achieve an attenuation of −6 dB. As in FIG. 5, certain taps 102 are opened (as represented by an “O”) and other taps 102 are left closed (as represented by an “⊗”). By opening the taps 102, a signal path 110 is created. As will be appreciated, by using the taps 102, coarse adjustments to the input signal can be achieved. In between each of the settings, fine adjustments are made through the RFB network 84, as previously discussed. Once the signal is set to 0 dB, the R2R ladder 80 is bypassed entirely, and an input transistor of the LNA 82 is used to actively terminate the LNA/TGC block 56 to properly match the impedance.

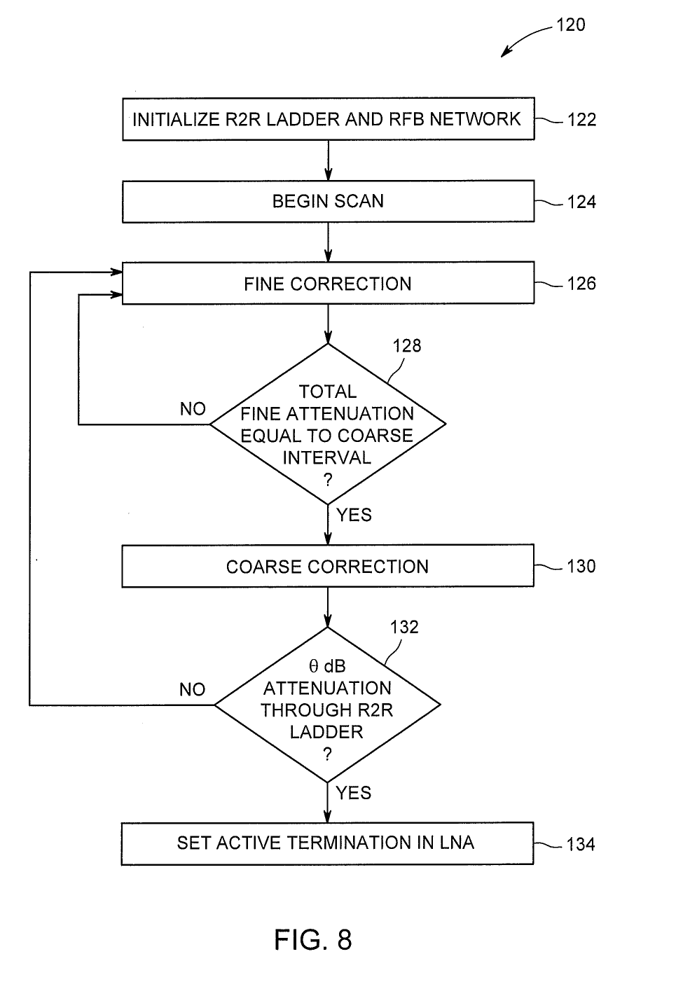

FIG. 8 is a flow chart summarizing the techniques described above, particularly with regard to FIGS. 5-8. That is, FIG. 8 provides the coarse and fine adjustments that may be made during a scan sequence, utilizing the LNA/TGC block 56, in accordance with embodiments of the disclosure. The first step in the process 120 is to initialize the R2R ladder 80 and RFB network 84 to provide maximum attenuation (e.g., −30 dB) to begin the scan, since the amplitude of the scan signal will be highest at the surface of the skin. The step is illustrated by block 122. Thus, the taps of the R2R ladder 80 are set such that the signal path includes the maximum number of R2R blocks 100 in parallel, as illustrated in FIG. 5, and the maximum number of resistors in the RFB network 84 are employed. Once the R2R ladder 80 and RFB network 84 are initialized, the scan may begin, as indicated in step 124. Once the signal is transmitted through the LNA/TGC block 56, the fine gain adjustments to the amplitude are made such that the gain of the input signal is slowly decremented in fine intervals (e.g., −0.3 dB), as indicated in block 126, utilizing a counter decremented shift clock in the RFB network 84. The fine interval adjustments are made until the total attenuation is equal to the coarse interval (e.g., −6 dB), as indicated by the decision block 128. In the present example, this may mean 20 steps of feedback cycles through the RFB network 84, for instance. Once the total attenuation through the feedback loop is equal to the coarse interval adjustment, the R2R ladder 80 is adjusted to coarsely attenuate the gain by the coarse interval adjustment, as indicated in block 130. That is, the taps 102 of the R2R ladder 80 are set such that one of the R2R blocks 100 is removed from the signal path through the R2R ladder 80 and the resistors in the RFB network 84 are reset to maximum attenuation. Thus, in the present example, the attenuation of the gain would be set to −24 dB through the R2R ladder 80 and the fine interval adjustments through the RFB network 84 would begin again until once again, the total attenuation through the feedback loop is equal to the coarse interval adjustment. This process is repeated until the R2R ladder 80 is set to 0 dB or no attenuation and the R2R blocks 100 are bypassed entirely, as indicated in block 132. After 1 final iteration of fine adjustment through the RFB network 84, active termination through the input transistor of the LNA 82 may then be utilized to more accurately match the impedance of the probe 12, as indicated in block 134.

As may be appreciated, the feedback resistors in the RFB network 84 may not always be incremented by exactly 20 steps due to lithography errors. The R2R ladder taps 102 may not exactly result in a 6 dB change, and therefore, the number of steps may vary as the taps 102 are changed. Calibration to adjust for the number of steps needed for every tap change in the R2R ladder 80 may be desirable. Once the calibration determines the number of steps required, these “stop” values may be recorded in registers. The start value is always the value that the feedback resistor resets back to prior to incrementing up to approximately 20 steps, or 6 dB.

This written description uses examples to disclose the present techniques, including the best mode, and also to enable any person skilled in the art to practice the techniques, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system comprising:
a low noise amplifier;
a coarse attenuation circuit coupled to an input of the low noise amplifier and configurable to attenuate an input signal by a coarse attenuation interval;
a fine attenuation circuit coupled in feedback with the low noise amplifier and configurable to attenuate the input signal by a fine attenuation interval, wherein the fine attenuation interval is less than the coarse attenuation interval.

2. They system of claim 1, wherein the coarse attenuation circuit comprises a plurality of resistive blocks arranged in parallel, wherein each of the resistive blocks may be configurably added to and removed from a signal path coupled to the input of the low noise amplifier.

3. The system of claim 2, wherein the resistive blocks are coupled to one another via a plurality of programmable switches.

4. The system of claim 2, wherein the coarse attenuation circuit is configurable to be bypassed such that no coarse attenuation to a signal path coupled to the input of the low noise amplifier is provided.

5. The system of claim 4, wherein an input impedance of the low noise amplifier provides termination to the coarse attenuation circuit when the coarse attenuation circuit is bypassed.

6. The system of claim 1, wherein the coarse attenuation interval is approximately −6 dB.

7. The system of claim 1, wherein the fine attenuation circuit comprises a plurality of feedback resistors.

8. The system of claim 1, wherein the system comprises an ultrasound system having a probe, and wherein the low noise amplifier, the coarse attenuation circuit and the fine attenuation circuit are contained within the probe.

9. A system comprising:
an ultrasound probe comprising:
a low noise amplifier;
an R2R ladder coupled to an input of the low noise amplifier and configured to provide coarse gain control through the low noise amplifier; and
a resistive feedback network coupled in feedback with the low noise amplifier and configurable to provide fine gain control through the low noise amplifier; and
a console communicatively coupled to the ultrasound probe and configured to facilitate ultrasound image collection and processing in conjunction with the ultrasound probe.

10. The system of claim 9, wherein the R2R ladder is configured to attenuate a scan signal in coarse attenuation intervals.

11. The system of claim 9, wherein the resistive feedback network is configured to attenuate an input signal by a fine attenuation interval.

12. The system of claim 9, wherein the R2R ladder comprises:
a plurality of resistive blocks; and
a plurality of taps coupled to the plurality of resistive blocks and configured to couple each of the plurality of resistive blocks in parallel.

13. The system of claim 12, wherein the R2R ladder comprises 5 resistive blocks and wherein each resistive block is configured to attenuate an input signal by approximately −6 dB.

14. The system of claim 9, wherein the low noise amplifier comprises an input transistor and wherein the system is configured to match an input impedance to a probe impedance.

15. The system of claim 14, wherein the R2R ladder is used to provide passive termination at the beginning of a scan sequence, and wherein the input transistor is used to provide active termination at the end of the scan sequence.

16. The system of claim 9, further comprising a capacitive feedback network arranged in feedback with the low noise amplifier and configured to adjust a bandwidth of an input signal.

17. The system of claim 9, wherein the fine gain control controls the gain through the low noise amplifier at at least an order of magnitude less than the coarse gain control controls the gain through the low noise amplifier.

18. A method for performing an ultrasound scan, comprising:
initializing a fine gain correction circuit and a coarse gain correction circuit to provide maximum attenuation of a scan signal;
adjusting, using the fine gain correction circuit, an attenuation of the scan signal by a fine attenuation increment until a total attenuation of the scan signal by fine attenuation increments is approximately equal to a coarse attenuation increment; and
adjusting, using the coarse gain correction circuit, the attenuation of the scan signal by the coarse attenuation increment.

19. The method of claim 18, wherein after adjusting the attenuation of the scan signal by the coarse attenuation increment, the method further comprises:
resetting the fine gain correction circuit for maximum attenuation; and
adjusting, using the fine gain correction circuit, the attenuation of the scan signal by the fine attenuation increment until the total attenuation of the scan signal by the fine attenuation increments is approximately equal to the coarse attenuation increment.

20. The method of claim 19, comprising repeating the steps of the method until the attenuation of the scan signal is equal to approximately zero.

21. The method of claim 20, comprising setting an active termination, utilizing a transistor of a low noise amplifier, after the attenuation of the scan signal is equal to approximately zero.

22. The method of claim 18, wherein adjusting, using the fine gain correction circuit, comprises adjusting, using a feedback resistor network, an attenuation of the scan signal by approximately −0.3 dB until a total attenuation of the scan signal is approximately equal to 6 dB.
